(19) United States
(12) Patent Application Publication
(10) Pub. No.: US 2021/0402097 A1
(43) Pub. Date: Dec. 30, 2021

(54) AUTO-INJECTOR WITH UNIFORM PRESSURE EXERTION OF A PRIMARY CONTAINER

(71) Applicant: Aktivax, Inc., Broomfield, CO (US)

(72) Inventors: Amir Genosar, Broomfield, CO (US); Matthew Gilbert Seehausen, Westminster, CO (US); Jason William Fox, Greenwood Village, CO (US); Richard Wayne LeVaughn, Arvada, CO (US); Andrew David Fish, Monument, CO (US); Eric William Sugano, Arvada, CO (US)

(21) Appl. No.: 17/360,850

(22) Filed: Jun. 28, 2021

Related U.S. Application Data
(60) Provisional application No. 63/044,896, filed on Jun. 26, 2020.

(51) Int. Cl. A61M 5/20; A61M 5/32

(52) U.S. Cl. CPC A61M 5/2053; A61M 5/32; A61M 5/2033

(57) ABSTRACT

Parenteral delivery of a beneficial agent using an auto-injector configured for reduction of forces acting on a primary container for the beneficial agent during activation of the auto-injector. The primary container may be disposed within a pressure chamber of the auto-injector such that an increased pressure within the pressure chamber during activation is applied to a stopper of the primary container and an external surface of a sidewall of the primary container. In turn, a pressure differential between a containment volume of the primary container and the pressure chamber may be reduced or eliminated. In turn, forces acing on the primary container in response to the pressure increase in the pressure container may be reduced or eliminated. Configurations are also provided for improved relative movement between a stopper and a sidewall of the primary container for more reliable injection.

### CROSS-REFERENCE TO RELATED APPLICATIONS

The present application claims benefit of priority to U.S. Provisional Patent Application No. 63/044,896, entitled “AUTO-INJECTOR UTILIZING A PRESSURE CHAMBER TO EXERT UNIFORM PRESSURE ON RIGID PRIMARY DRUG CONTAINERS” and filed on 26 Jun. 2021, which is specifically incorporated by reference herein for all that it discloses or teaches.

### FIELD

The present disclosure pertains to the field of parenteral delivery of a beneficial agent, and more particularly to prefilled auto-injection devices powered by an energy source.

### BACKGROUND

Auto-injectors are devices used for parenteral delivery of one or more beneficial agents to subjects. Auto-injectors typically enable injection parameters, such as injection depth, injection force, injection volume, and duration of injection, to be predetermined and independent of user input. Auto-injectors may be supplied as ready-to-use products that may be prefilled with a fixed dose of one or more beneficial agents. In some examples, auto-injectors may be used for administering life-saving emergency treatments such as epinephrine for anaphylaxis by either intramuscular or subcutaneous injection. Other examples in which auto-injectors may be utilized include civilian or military emergency medical treatments. Auto-injectors provide automation of at least some of the steps of the injection procedure such that use of an auto-injector to inject a beneficial agent may reduce the potential for user errors and may allow injection to be performed quickly and reliably outside of healthcare settings by lay persons who are not medical professionals.

Auto-injectors commonly comprise a needle that penetrate the subject's tissue and function as a conduit for injecting the beneficial agent into the subject's tissue. In other examples, jet auto-injectors (jet injectors) do not comprise a needle, but instead produce a narrow, high-pressure stream of the beneficial agent that can penetrate into subject tissue by force of the stream of the beneficial agent alone. In any regard, auto-injectors may utilize an energy source, such as springs, compressed gas cylinders, or the like, to power needle insertion and/or depositing of beneficial agents into a subject's tissue. Regardless of the type of auto-injector, activation of the auto-injector by an energy source may result in substantial forces acting on components of the auto-injector.

A beneficial agent may be stored inside the auto-injector body in a primary container. The primary container may be made of glass or polymer. The primary container may be integral to the auto-injector and non-replaceable, or may take the form of a replaceable container. In some examples, the primary container may also be in the form of a pre-filled syringe with an integrated needle.

Auto-injector primary containers may be rigid, having a fixed shape. Alternatively, primary drug containers for auto-injectors may be flexible, having a pliable shape with an adjustable volume for containing a beneficial agent. Rigid primary containers may comprise hollow, cylindrical bodies including a sidewall. Rigid primary containers may include a moveable plunger that defines a containment volume within the cylindrical body in which the beneficial agent is contained. The plunger may bear upon an interior surface of the sidewall of the cylindrical body to fluidly seal the beneficial agent inside the containment volume. In addition, movement of the plunger relative to the cylindrical body may act to reduce the containment volume and to expel the beneficial agent from the primary container and/or auto-injector. Movement of the plunger relative to the cylindrical body may occur when a force is applied from an activated energy source to create a pressure differential between a beneficial agent in the containment volume of the container and the injection site or an environment exterior to the container.

In this regard, the primary container may resemble that of a standard syringe where the moveable plunger is moveable relative to a cylindrical body to pressurize a containment volume containing the beneficial agent to expel the beneficial agent through a needle. The plunger may be moved in response to application of a force resulting from activation of the energy source. For instance, pressurized fluid may be used to apply a force to move the plunger and expel the beneficial agent. Auto-injectors that utilize compressed gas cylinders as an energy source may comprise a pressure chamber that remains sealed from outside environment while the beneficial agent is being expelled from the primary container. In turn, activation of the energy source may increase the pressure in the pressure chamber relative to an exterior environment of the auto-injector in order to develop a pressure differential that drives the injection. Prior proposed auto-injectors may apply a force resulting from the pressure differential directly to a plunger of a primary drug container or may apply the force to a plunger of a primary drug container by intermediate elements that may also function to regulate the force applied to the plunger.

### SUMMARY

The present disclosure provides an auto-injector for administering a beneficial agent. The auto-injector includes a pressure chamber and a primary container. The primary container includes a barrel defining a rigid sidewall with an inner surface and an exterior surface. The barrel is disposed within the pressure chamber. For example, a portion of, a majority of, or an entirety of the barrel may be disposed within the pressure chamber (i.e., within a boundary of the pressure chamber such that no part of the barrel is exposed to a differential in pressure between respective portions of the barrel). A stopper is disposed in the barrel and sealingly engaged with the inner surface to define a containment volume within the primary container. The containment volume induces a beneficial agent. An increase in pressure within the pressure chamber results in an increase in a first pressure in the pressure chamber acting uniformly on the stopper and the exterior surface to increase a second pressure in the containment volume creating a pressure differential relative to ambient pressure of an exterior environment of the auto-injector to cause the stopper to move relative to the inner surface to reduce the containment volume and expel the beneficial agent from the primary container. However, as the barrel of the primary container may be disposed within the pressure chamber and experience no pressure differential with respect thereto, the primary container may experience significantly decreased forces acting thereon during activation (e.g., as compared to a primary container having a sidewall defining a portion of the pressure chamber).

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Other implementations are also described and recited herein.

### BRIEF DESCRIPTION OF THE DRAWINGS

FIG. 1 illustrates a schematic view of an example auto-injector.

FIGS. 2a-2c illustrate an example of an auto-injector in a pre-activation, mid-activation, and post-activation configuration.

FIG. 3 illustrates an exploded view of an example connector for engagement with a cartridge in the example shown in FIGS. 2a-2c.

FIGS. 4a-4c illustrate another example of an auto-injector in a pre-activation, mid-activation, and post-activation configuration.

FIG. 5 illustrates an exploded view of an example connector for engagement with a cartridge in the example shown in FIGS. 4a-4c.

FIGS. 6a-6b illustrates a detailed view and an exploded view of an example connector for engagement with a cartridge.

FIGS. 7a-7c illustrate detailed views of an example of a cartridge in various stages of injection of a beneficial agent.

FIGS. 8a-8c illustrate detailed views of an example of a cartridge in various stages of injection of a beneficial agent.

FIGS. 9a-9b show magnified views of FIGS. 8a-8b to illustrate axial stretching and radial contraction of a stopper, and concomitant reduction of friction with a sidewall

### DETAILED DESCRIPTION

While the invention is susceptible to various modifications and alternative forms, specific embodiments thereof have been shown by way of example in the drawings and are herein described in detail. It should be understood, however, that it is not intended to limit the invention to the particular form disclosed, but rather, the invention is to cover all modifications, equivalents, and alternatives falling within the scope of the invention as defined by the claims.

The present disclosure relates to auto-injector devices for parenteral delivery of beneficial agents. The beneficial agent is stored in a primary container. The primary container may comprise a sidewall defining a portion of a containment volume in which a beneficial agent is provided. The sidewall may comprise cylindrically shaped rigid barrel housed inside the auto-injector body (e.g., within a pressure chamber of the auto-injector). The primary container may contain the beneficial agent in the containment volume defined by the sidewall and a stopper that serves to seal the containment volume. The stopper may also serve as a moveable plunger that may move along the sidewall within the barrel to expel the beneficial agent from the primary container (e.g., through a port of the primary container that may be in fluid communication with an administration device of the auto-injector). Upon movement of the stopper relative to the barrel, the containment volume may be reduced and the beneficial agent may be expelled from the primary container at the time of injection.

In the present disclosure, the sidewall of the barrel as well as an external surface of the stopper that is opposite to an inner surface (e.g., the surface in contact with the beneficial agent) defining a boundary of the containment volume may be disposed within (e.g., entirely within) the pressure chamber. In turn, the primary container (e.g., including the external surface of the stopper and an external surface of the sidewall of the primary container) may be exposed to a pressure within the pressure chamber. This arrangement enables uniform pressure to be applied to the exterior surface of the sidewall as well as the external surface of the stopper. As the stopper may be moveable within the primary container, a corresponding pressure increase experienced in the pressure chamber may also be experienced within the containment volume of the primary container. Thus, a pressure differential between the pressure of the pressure chamber and a pressure within the containment volume may be reduced or even eliminated upon activation of an energy source of the auto-injector. That is, the forces exerted on the interior surface and the exterior surface of the barrel may be in near balance during the injection, limiting or eliminating stresses acting on the walls of the barrel. However, the pressure in the containment volume may be lower than the pressure in the pressure chamber due to friction between the interior surface of the sidewall and the stopper. Furthermore, as the containment volume is provided within the pressure chamber, which may be at an increased pressure upon activation of the auto-injector, a pressure differential may exist between the containment volume and an exterior environment of the auto-injector which may result in the beneficial agent being expelled from the containment volume (e.g., through a port in fluid communication with an administration device). In turn, the beneficial agent is expelled from the auto-injector but the primary container is not subjected to the potentially large forces generated in response to activation of the auto-injector.

The balancing of forces across the sidewall of the containment volume may facilitate a number of distinct advantages relative to other arrangements proposed for auto-injectors. For instance, auto-injectors may act to expel a beneficial agent by directly pressurizing the primary container that is provided external to a pressure chamber or forms a portion of the pressure chamber. In the latter auto-injectors, the force is applied directly to the plunger such that an external surface of the plunger forms a portion of the boundary of the pressure chamber. As the plunger travels inside the primary container in response to a force acting on the plunger, a portion of the sidewall of the primary container above the plunger may be exposed. In turn, the sidewall of the primary container thus also forms a portion of the boundary of the pressure chamber as the plunger is advanced in the primary container. Simultaneously, the containment volume (i.e., portion of the primary container below the plunger) becomes pressurized, also exerting pressure on the interior of the sidewall that is higher than the pressure on the exterior of the sidewall. Because the sidewall of the primary container and the external surface of the plunger comprise a boundary of the pressure chamber, these structures experience a force resulting from the pressure differential between a pressure within the pressure chamber and a pressure external to the pressure chamber. That is, an internal surface of the primary container may be exposed to an increased pressure from the pressure chamber while an exterior surface of the primary container may be exposed to lower, ambient pressure. As noted above, in many examples, this resulting force may be relatively large. In the former auto-injector designs, the primary container is completely external to the pressure chamber, wherein the pressure is transmitted to the plunger by mechanical means, commonly by a rigid rod, rather than directly by gas pressure being applied to the plunger. In such auto-injectors, the portion of the sidewall defining the containment volume, but not the portion of the sidewall above the plunger, experiences a pressure differential.

The ability of primary containers to withstand forces acting on the sidewall of the primary container may be limited by the characteristics of materials from which the primary container is made. In the field of pharmaceutical packaging, the primary consideration in the selection of primary container material is typically the chemical properties of the material such as, for example, barrier properties, impurities, and potential for physical and chemical interactions with beneficial agents. Inappropriate selection of a material with unsatisfactory chemical properties could be detrimental to the clinical efficacy of the beneficial agent. For this reason, primary container materials may be limited to glass and/or specialized polymers that are chemically inert with respect to the beneficial agent. Accordingly, because of the focus on chemical properties of primary container materials, such specialized materials may not provide robust physical properties sufficient to reliably withstand stresses within the material resulting from the potentially large forces experienced during auto-injector activation. For example, such materials may result in either brittle or soft containers. Either brittle or soft containers may be disadvantageous when exposed to large forces in the presence of a large pressure differential across an interior and exterior of a primary container.

While the physical properties of a primary container are not necessarily a concern in other applications (e.g., traditional syringes manipulated by a human user), primary containers in auto-injectors may experience significant stresses in response to forces acting on the primary container that can affect the performance of the primary container. Such high stresses acting on the primary container pose a significant risk to auto-injectors reliability. For instance, high forces acting on a primary container sidewall resulting in instances where the sidewall of the primary container forms a boundary of a pressure container may lead to mechanical failure (e.g., breaking, cracking, separation, etc.) of the container. This may result in a failed or an incomplete injection. Even in instances where the sidewall does not mechanically fail, the sidewall may deform in response to the forces acting on it, which may lead to a loss of seal between the plunger and the primary container, which may also result in a failed injection, an incomplete injection, or undesirable exposure of the beneficial agent.

In addition to the stresses that occur during the injection itself, primary containers may be weakened over time during normal handling, as auto-injectors are often intended to be carried and used outside of controlled healthcare environments. As a result, primary containers may be exposed to physical shock, extreme temperatures, extreme moisture conditions, or other environmental conditions that may further physically degrade the primary container material. Furthermore, such degradation may not become apparent until the auto-injector is used, when stresses incurred during activation of the auto-injector may result in a failure of a weakened primary container. Furthermore, increasingly stringent auto-injector reliability requirements implemented by regulatory bodies elevate the magnitude of this risk in terms of marketability.

Moreover, efforts to mitigate shortcomings in the physical property of the primary container may not be economically feasible or effective. For instance, use of exotic materials that provide desirable chemical and physical properties, if even available, may result in primary containers that are not economically feasible for use in auto-injectors. Moreover, attempts to reinforce the primary container such as by enclosing the container in a secondary or reinforcing container does not fully address these risks as the secondary container may also deform or fail. Further still, friction may be produced as a result of the interface of the primary container and secondary or reinforcing container, contributing additional risks to the integrity of the primary container. For instance, the primary container may chafe on the reinforcing container during storage of the auto-injector prior to activation. Moreover, a reinforcing container may impinge on the primary container, which may affect the ability for the plunger to smoothly move along a sidewall of the primary container. Further still, an elongated plunger that occupies an interior space of the container as the elongated plunger is advanced to reinforce the sidewall of the container as the elongated plunger is advanced within the primary container may be contemplated. However, such an elongated plunger may experience increased friction as the elongated plunger is advanced, resulting in increased force on the sidewall that may cause the elongated plunger to bind or experience increased friction, thus resulting in a potential for incomplete injection. Further still, incomplete contact between the elongated plunger and the sidewall may still expose the sidewall of the primary container to stresses, which may be concentrated or localized in areas of incomplete contact with the elongated plunger. Also, in auto-injectors that utilize intermediate elements in which the pressure chamber is isolated from the plunger, the plunger may be mechanically pushed by the intermediate element such that the primary container does not come into direct contact with the increased pressure in the pressure chamber. However, the pressure within the containment volume may still increase relative to a pressure external to the containment volume causing increased stresses in the sidewall of the primary container. Thus, in these approaches, a pressure on the interior of the sidewall of the primary container is experienced when it is pressurized by the plunger, resulting the susceptibility to the failure modalities noted above.

Therefore, the examples provided herein may facilitate an improved auto-injector design that mitigate the risk of failure to successfully perform an injection that stems from the limitations imposed by primary drug containers. The examples provided herein may use traditional primary container materials that exhibit beneficial chemical properties such as being chemically inert with respect to the beneficial agent. As such, the design contemplated in the present disclosure may provide an economically viable approach that does not add complexity to the injector design yet mitigates the risk of physical failure of the primary container during activation of an auto-injector. In the following disclosure, a schematic illustration of an example of an arrangement of a primary container is provided. Thereafter examples of auto-injector arrangements are described that may realize the advantages of the present disclosure with a reusable primary container that has reliable movement of a stopper in the primary container to reliably expel a beneficial agent therefrom.

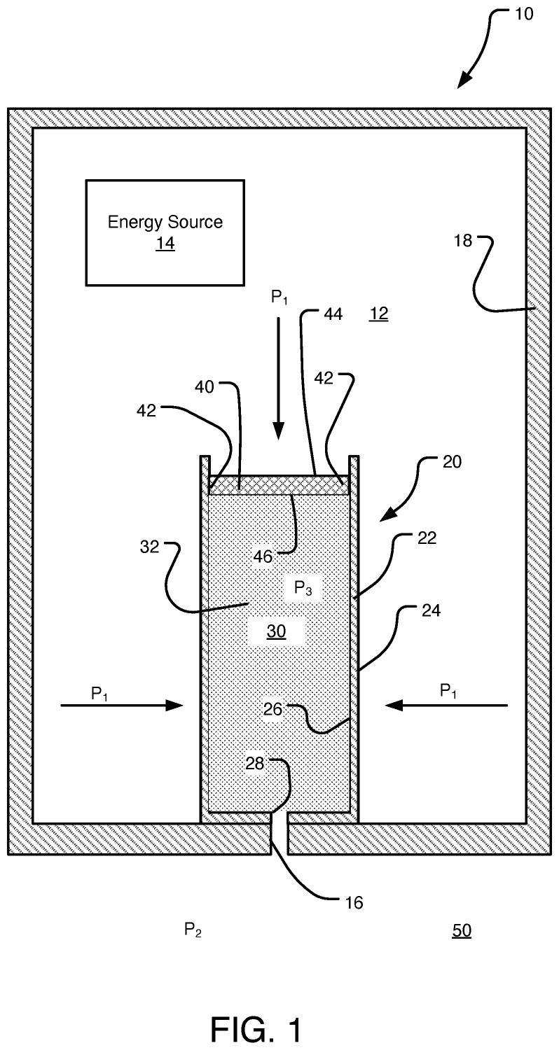

With reference to FIG. 1, an example of an auto-injector 10 is illustrated in a schematic representation. The auto-injector 10 may include a pressure chamber 12. The pressure chamber 12 may be defined by a pressure chamber wall 18. The auto-injector 10 may include an energy source 14 within the pressure chamber 12 that may be selectively activated to increase a pressure P₁ within the pressure chamber 12. The pressure chamber wall 18 may define a boundary of the pressure chamber 12 across which a pressure differential exists when the pressure chamber 12 is pressurized in response to activation of the energy source 14. In this regard, the wall 18 of the pressure chamber 12 may be a material selected to have physical properties capable of withstanding forces acting on the wall 18 during activation.

The energy source 14 may be any appropriate device or mechanism that is selectively activatable to cause an increase in the pressure P₁ in the pressure chamber 12 relative to an ambient pressure P₂ in an exterior environment 50. For instance, the energy source 14 may include a spring, compressed gas source, or other appropriate mechanism or device that may be selectively activated to increase the pressure P₁.

A primary container 20 is provided within the pressure chamber 12. The primary container 20 may include a sidewall 22. The sidewall 22 may define a rigid cylindrical structure. A stopper 40 may be disposed relative to the sidewall 22. The stopper 40 may provide a sealing surface 42 that provides a fluid seal between the stopper 40 and an inner surface 26 of the sidewall 22. In turn, a containment volume 30 may be defined by an inner surface 46 of the stopper and an inner surface 26 of the sidewall 22. The containment volume 30 may contain a beneficial agent 32.

The primary container 20 may include an outlet 28. The outlet 28 may be aligned or otherwise fluidly connected with a port 16 of the auto-injector 10. While not shown in FIG. 1, the port 16 may comprise or be in further fluid communication with an administration device (not shown) for administration of the beneficial agent 32 using the auto-injector 10. It may be appreciated that the administration device may be any appropriate administration device now known or later developed including a needle, jet nozzle, oral dispenser, topical dispenser, or other appropriate device or apparatus. Moreover, while not shown in FIG. 1, the administration device may be activatable in response to the activation of the energy source 14 to deploy the administration device from the auto-injector 10 (e.g., to expose a portion of the administration device to an exterior of a housing of the auto-injector). While examples of an administration device are illustrated in greater detail below, such examples are illustrative and not limiting.

Also, while FIG. 1 shows the primary container 20 as being distinct from the wall 18 of the pressure chamber 12, it may be appreciated that the primary container 20 may include any appropriate interface between the primary container 20 and the pressure chamber 12 so as to provide the primary container 20 in position within the pressure chamber 12 to facilitate expelling the beneficial agent 32 from the containment volume 30. For instance, the primary container 20 may be provided as an integral component of the wall 18 of the pressure chamber. Alternatively, a connector may be provided that interfaces the primary container 20 with the auto-injector 10 to dispose the primary container 20 in an appropriate arrangement within the pressure chamber 12. Examples of connectors are described in greater detail below. In any regard, the primary container 20 may be replaceable such that the auto-injector 10 may facilitate multiple uses with new primary containers 20 being provided subsequent to each activation of the auto-injector 10.

Of particular note, the sidewall 22 of the primary container 20 may be disposed within the pressure chamber 12. Accordingly, the sidewall 22 is provided within a boundary of the pressure chamber 12. While illustrated in FIG. 1 in a manner such the sidewall 22 is wholly disposed within the pressure chamber 12, it may be appreciated that a portion of or a majority of the sidewall 22 may be disposed within the pressure chamber 12. As such, an exterior surface 24 of the sidewall 22 may be exposed to the pressure P₁ within the pressure chamber 12. For example, upon activation of the energy source 14, pressure P₁ within the pressure chamber 12 may be elevated relative to the ambient pressure P₂ of the exterior environment 50. The pressure P₁ within the pressure chamber 12 may act uniformly on the primary container 20 including on the exterior surface 24 of the sidewall 22 as well as the exterior surface 44 of the stopper 40. As the stopper 40 may be moveable (e.g., axially along the cylindrical sidewall 22), the containment volume 30 may undergo a corresponding increase in pressure with the increase of pressure P₁. In turn, the containment volume 30 may also be at or near P₁. Accordingly, a pressure differential between the containment volume 30 and the pressure chamber 12 may be minimal or there may be no pressure differential between the inner surface 26 of the sidewall 22 of the containment volume 30 and an exterior surface 24 of the sidewall 22 exposed to the pressure chamber 12. However, due to friction between the stopper 40 and the cylindrical sidewall 22 at the sealing surface, the pressure in the containment volume 30 may be lower than pressure P₂. Furthermore, as the pressure P₁ in the pressure chamber 12 may be greater than the ambient pressure P₂ in the exterior environment 50, a pressure differential may exist between the containment volume 30 and the exterior environment 50. This may result in the beneficial agent 32 being expelled through the outlet 28 (and in turn, port 16). As the beneficial agent 32 is expelled, the stopper 40 may move relative to the sidewall 22 to reduce the containment volume 30. The stopper 40 may move distally relative to the sidewall 22 as the containment volume 30 is reduced. The stopper 40 may be configured to move axially along the rigid cylindrical sidewall 22. The stopper 40 may also be configured to maintain an orientation relative to the cylindrical sidewall 22 (e.g., to maintain the sealing surface 42 by maintaining the stopper 40 in an orthogonal orientation relative to the longitudinal axis of the cylindrical sidewall 22. Additional examples of a stopper configured to maintain sealing engagement with a cylindrical sidewall are described in greater detail below.

The reduction in the containment volume 30 may be in response to the pressure differential between the pressure P₁ experienced by the containment volume 30 and the ambient pressure P₂. Of note, as the stopper 40 is moved to expel the beneficial agent 32 from the containment volume 30, there continues to be very little or no pressure differential between the containment volume 30 and the pressure chamber 12. In turn, the sidewall 22 does not experience large forces resulting from a pressure differential across the inner surface 26 and the exterior surface 24 of the sidewall 22. Thus, the sidewall 22, by virtue of having balanced pressure exposure on the inner surface 26 and exterior surface 24 as the sidewall 22 is disposed within the pressure chamber 12, does not undergo a large force acting on the sidewall 22. In turn, stresses within the sidewall 22 may be reduced or eliminated, thus reducing the potential for mechanical failure of the sidewall 22 or the sealing surfaces 42.

In contrast to the proposed arrangements described above in which the sidewall of a primary container forms a boundary of a pressure chamber of an auto-injector, the arrangement shown and described in relation to FIG. 1 provides advantages in that the sidewall 22 of the primary container 20 does not experience forces resulting from a pressure differential between the containment volume 30 and an exterior of the container volume 30. That is, because the primary container 20 does not form a boundary of the pressure chamber 12, the primary container 20 does not experience a pressure differential between the inner surface 26 and the exterior surface 24 that results in stresses in the sidewall 22 of the primary container 20. Rather, the primary container 20, being disposed within the pressure chamber 12, experiences uniform pressure being applied across the sidewall 22 of the primary container 20. Thus, the beneficial agent 32 may be expelled from the primary container 20 in response to the increased pressure P₁ without introducing forces in the sidewall 22 of the primary container 20 that may lead to mechanical failure of the sidewall 22 or deflection of the sidewall 22 relative to the stopper 40 compromised sealing surfaces 42. The present disclosure further relates to attachment to a port of a cartridge for a beneficial agent. The present disclosure further relates to an arrangement for reducing the friction of a plunger moving in a barrel.

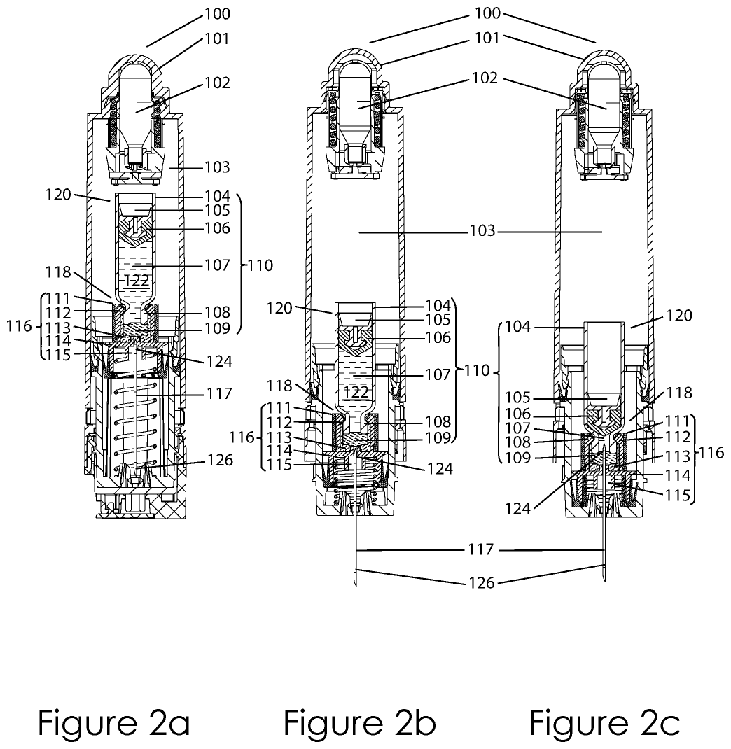

FIGS. 2a-2c illustrate another example arrangement of an auto-injector 100 for administering a beneficial agent 107 to a subject. The auto-injector 100 comprises a housing 101 which defines a pressure chamber 103. The auto-injector includes an energy source 102. While not described in detail herein, the energy source 102 and/or activation methodologies for the energy source 102 may be provided according to any of the teachings of U.S. Pat. No. 10,716,901, and/or U.S. Pat. No. 11,001,435, which are incorporated by reference herein in their entirety.

The auto-injector 100 also includes an administration device 117. As noted above, while the administration device 117 is shown and described below as including a needle, the administration device 117 may be any appropriate administration device for expelling the beneficial agent 107 including, for example, a jet nozzle, a topical applicator, an oral dispenser, or the like.

The auto-injector 100 may include a primary container for containing the beneficial agent 107. In the example shown in FIGS. 2a-2c, the primary container comprises a cartridge 110. The cartridge 110 disposed in the pressure chamber 103. The cartridge 110 comprises a sidewall, which in the illustrated example may comprise a rigid cylindrical barrel 104 with a distal end 118 and a proximal end 120. The proximal end 120 may be configured to expel the beneficial agent 107. The cartridge 110 further comprises a stopper 106 moveably disposed in the barrel 104. The stopper 106 seals against an inner surface of the barrel 104 to define a containment volume 122 between the stopper 106 and the barrel 104. The beneficial agent 107 is disposed in the containment volume 122.

The energy source 102 may be configured to be activated when the auto-injector is used to pressurize (e.g., increase the pressure within) the pressure chamber 103. The administration device 117 may be configured to transfer the beneficial agent 107 as it is expelled from the cartridge 110 and to administer the beneficial agent 107 to the subject. In some embodiments, the cartridge 110 may comprise a plurality of stoppers or other apparatus to create a plurality of compartments for storing multiple beneficial agents and/or for separately storing a dry (lyophilized or freeze dried) beneficial agent and its diluent for reconstitution at the time of injection. In such multi-compartment embodiments, the barrel 104 and/or stopper 106 may include at least one bypass (e.g., a wider section that permits fluid communication between adjacent compartments when occupied by an intermediate stopper). For instance, U.S. Pat. No. 8,092,421, which is incorporated by reference herein, teaches such an arrangement in a cartridge intended for use in beneficial agent delivery devices.

The cartridge 110 disposed in the pressure chamber 103 may be configured such that pressure within the pressure chamber 103 acts on the stopper 106 to pressurize the beneficial agent 107 within the containment volume 122. In addition, as described above in FIG. 1, the pressure within the pressure chamber 103 also acts on an external surface of the barrel 104. Because the pressure of the beneficial agent 107 may be equalized with the pressure acting on the external surface of the barrel 104, any forces exerted by the beneficial agent to an internal surface of the barrel 104 may be reduced or eliminated while the beneficial agent 107 is expelled from the cartridge 110 through the administration device 117.

In some embodiments the cartridge 110 further comprises a piston 105 moveably disposed in the barrel 104. The piston 105 may be disposed relative to the stopper 106. For instance, the piston 105 may be provided distally relative to the stopper 106. The piston 105 may also seal against the inner surface of the barrel 104 to assist in reducing the likelihood that the pressurized fluid from the pressure chamber 103 bypasses the seal of the piston 105 and reaches the stopper 106. Also, the piston 105 may help to maintain the orientation of the stopper 106 within the barrel 104 as the stopper 106 moves distally relative to the barrel 104. By maintaining the orientation of the stopper 106 relative to the barrel 104, the sealing engagement of the stopper 106 relative to the barrel 104 may be maintained to maintain the integrity of the containment volume 122.

In some examples, the stopper 106 may be made of an elastomeric material that is relatively soft and/or flexible. In turn, when experiencing varying levels of friction inside the barrel 104 during distal movement of the stopper 106 in the barrel 104, the stopper 106 may be prone to shifting out of perpendicular engagement to the barrel 104 and a loss of seal may occur. As such, a piston 105 may comprise a more rigid material relative to the stopper 106. In turn, the piston 105 may convert the force resulting in movement of the stopper 106 and piston 105 to mechanical force on the stopper 106 to stabilize the stopper 106 during activation of the auto-injector 100. In some embodiments, the interior surface of the barrel 104, a sealing surface of the stopper 106, and/or a sealing surface of the piston 105 may be coated with silicone oil, in a process known in art as siliconization. Siliconization may be provided in order to lubricate the respective surfaces which undergo relative movement thus ensuring smooth relative movement therebetween.

In some embodiments the energy source 102 may comprise one of compressed gas or liquefied gas, as is known in the art. Gas pressure sources consist of a canister made of high strength material such as steel, an actuating mechanism that allows the gas to be released and may also include a regulator to control the release rate and amount of released gas. Compressed gasses commonly utilized as energy sources in auto-injectors and other gas-driven devices include carbon dioxide, nitrogen, and nitrous oxide. Gas can also be compressed to its vapor pressure at which point it transitions to liquid form and occupies a smaller volume, allowing a smaller gas canister to be used. Common liquefied gas propellants include hydrocarbons, fluorocarbons, and ethers.

In one embodiment, the auto-injector 100 comprises an administration device 117 that is a needle configured on a first end 124 to establish fluid communication with the beneficial agent 107 inside the cartridge 110 and configured on a second end 126 to penetrate the subject's tissue. At the time of activation, the first end 124 of the needle may penetrate into the containment volume 122, which may provide a flow path for the beneficial agent 107 such that the beneficial agent 107 may travel through the administration device 117 from the cartridge 110 into the subject's tissue via the second end 126 in response to the pressure differential between the containment volume 122 and an ambient pressure in the subject's tissue.

In some embodiments, the administration device 117 is one of a needle, a micro-needle, an array of needles or micro-needles, a jet injector nozzle, a nasal nozzle, a dispenser, a connector to any of the foregoing, or any other appropriate administration device. Whereas a standard needle may be used for intramuscular or subcutaneous injections and can deliver an injection through clothing, injection characteristics of other administration devices may be preferred, depending on a particular treatment. For smaller injection volumes, subcutaneous injections and cutaneous injections, a micro-needle or an array of hollow or solid micro-needles can be used. Needle arrays can provide for an easier self-administration, less painful to the subject, and do not generate hazardous waste. Jet injectors do not use a physical conduit for the beneficial agent, but instead rely on the kinetic energy of the beneficial agent itself. Jet injectors may shape the beneficial agent into a narrow stream and propel it at high velocity sufficient to penetrate through the skin of the subject. A nasal nozzle can be used to deliver the beneficial agent into the nasal cavity of the subject, where the beneficial agent is absorbed into the blood stream through mucous membranes. It can also be used to deliver the beneficial agent into the subject's brain, bypassing systemic circulation by depositing the beneficial agent onto the olfactory epithelium where it is absorbed by olfactory and trigeminal nerves. Yet in other embodiments the administration device is a connector configured for interfacing with a needle, a micro-needle, an array of needles or micro-needles, a jet injector nozzle, a nasal nozzle, a dispenser. This arrangement enables the auto-injector to be adapted for any of these delivery routes, allowing the administration method to be selected just prior to injection, and also simplifying manufacturing of auto-injectors for beneficial agents intended for multiple routes of delivery.

The components of the auto-injector arrangement that serve to prevent the auto-injector 100 from activating during storage, prepare the auto-injector 100 for injection, activate the auto-injector 100, operate the energy source 102 to release gas upon activation, and/or perform the injection may be provided according to any of the teachings of U.S. Pat. No. 10,716,901, and incorporated by reference in its entirety herein. The '901 Patent discloses the auto-injector arrangement states in terms of configurations of these components before, during, and after injection.

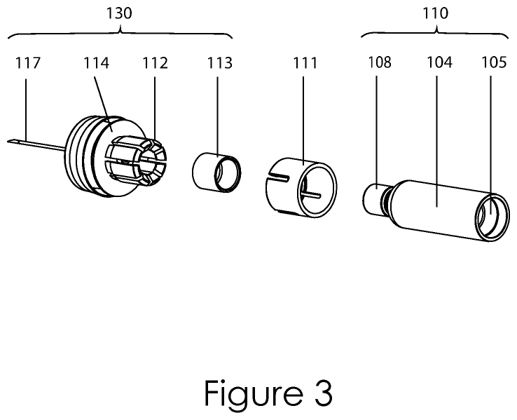

As noted above, a primary container may interface with the auto-injector 100 by way of an interface mechanism or connector. As illustrated in FIGS. 2a-2c and FIG. 3, the primary container in the form of the cartridge 110 may interface with the auto-injector 100 by way of a connector 116. The connector 116 may comprise a body 114 having a distal end and proximal end. An opening 115 may be provided in the body 114 extending between the proximal end and distal end of the body 114. The opening 115 may be configured for receiving the administration device 117. A sealing surface 113 may be provided surrounding the opening 115.

In the example shown in FIGS. 2a, 2b, 2c, and 3, the cartridge 110 may be engaged by a plurality of latch arms 112 (best seen in FIG. 3). The latch arms 112 may be disposed at the distal end of the connector 116 and extend distally with respect to the body 114. The latch arms 112 may be configured to deflect radially as the cartridge 110 is advanced proximally. In turn, a locking ring 111 may be provided that may concentrically slide relative to the cartridge 110 to surround the latch arms 112 to secure the cartridge 110 thereto.

For instance, the cartridge 110 may comprise a flange 108 at a distal end thereof. A septum 109 may be disposed relative to flange 108 and configured to seal the beneficial agent 107 within the containment volume 122 of the cartridge 110. In an example, the septum 109 may be penetrated by the first end 124 of a needle of the administration device 117 to establish a fluid path for the administration of the beneficial agent 107. In any regard, the flange 108 may be engaged by the set of latch arms 112 of the connector 116. Specifically, the latch arms 112 may be configured to (in the absence of the locking ring 111) deflect radially to accept the flange 108 such that the latch arms 112 engage the flange 108 once the flange 108 is engaged with the latch arms 112. That is, the latch arms 112 may include engagement structures that allow for radial deflection of the latch arms 112 as the flange 108 is engaged therewith, yet the engagement structures may restrict disengagement of the flange 108 once engaged therewith. Specifically, the latch arms 112 may extend substantially parallel to the barrel 104 of the cartridge 110. The latch arms 112 may be thickened at the distal ends of the latch arms 112 such that the latch arms 112 define a shoulder that extends radially inward. Upon outward radial deflection of the latch arms 112, the flange 108 may be advanced distally relative to the shoulder of the latch arms 112. Once the flange 108 passes the shoulder of the latch arms 112, the latch arm 112 may be biased radially inward such that the shoulder engages a proximal surface of the flange 108 to resist removal of the cartridge 110 from the connector 116 (e.g., to resist relative axial movement between the cartridge 110 and the connector 116). A sealing surface 113 (e.g., an elastomeric material or the like) may be disposed between the connector body 114 and the cartridge 110 when engaged by the latch arms 112 to provide a fluid seal between the connector body 114 and the cartridge 110. Specifically, the sealing surface 113 may be positioned between the latch arms 112 and the flange 108, surrounding the opening 115 and the septum 109 to secure the septum 109 in place and to provide a seal at the interfaces of the flange 108 with the septum 109 and the septum 109 with the opening 115.

Moreover, a locking ring 111 may be advanced concentrically relative to the cartridge 110 to dispose the locking ring 111 about the latch arms 112 once engaged with the flange 108. The locking ring 111 may further resist radial deflection of the latch arms 112 away from the flange 108 once engaged with the flange 108 to prevent removal of the flange 108 from engagement with the latch arms 112.

The connector 116 serves to interface the cartridge 110 with the auto-injector 100 by disposing the cartridge 110 in a relative position to the administration device 117. The connector 116 provides a secure coupling of the cartridge 110 that may be facilitated during an automated manufacturing process into a pre-use configuration (e.g., shown in FIG. 2a). The connector 116 may also maintain the cartridge 110 relative to the administration device 117 to facilitate fluid communication between the containment volume 122 of the cartridge 110 and the administration device 117 during use of the auto-injector 100 (e.g., during injection). As is known in the art, primary containers such as the cartridge 110 may be manufactured from pharmaceutical packaging materials in a separate process. In turn, the cartridge 110 may be later integrated into drug delivery devices such as the auto-injector 100.

A proximal end of the connector body 114 comprises an opening that may accepts the administration device 117 and positions the septum 109 of the cartridge 110 in position to be engaged by the administration device 117. The opening 115 of the connector 116 may assist in providing straight motion of the administration device 117 through the septum 109 and into the containment volume 122 containing the beneficial agent 107.

FIG. 2b illustrates an arrangement of the auto-injector 100 after it has been activated and before the injection of the beneficial agent 107 into a subject. The energy source 102 may pressurize the pressure chamber 103. In response to the pressurization, the connector 116, cartridge 110, and administration device 117 may be moved relative to the auto-injector housing 101 to expose the second end 126 of the needle beyond the auto-injector housing 101 (e.g., into the subject's tissue). The movement of the administration device 117 may also move the cartridge 110 which is coupled to the administration device 117 by the connector 116 in the same direction relative to the auto-injector housing 101. However, when the administration device 117 reaches the limit of its range of motion, the connector 116 and the cartridge 110 may continue to move relative to the administration device 117, overcoming a biasing force applied by a spring or the like, under influence of the increased pressure in the pressure chamber 103. Thus, the connector 116 and cartridge 110 may continue to move relative to the administration device 117 such that the first end 124 of the needle of the administration device 117 pierces the septum 109 to establish fluid communication between the administration device 117 and the containment volume 122. When fluid communication is established between the containment volume 122 and an exterior environment, a pressure differential between the pressure chamber 103 and an ambient pressure of the exterior environment may result in a force on the piston 105, which may transfer the force to the stopper 106. In turn, the beneficial agent 107 is pressurized and expelled via the administration device 117. The pressure of the pressure chamber 103 is also applied at the exterior surface of the barrel 104, countering the pressure of the beneficial agent 107 on the interior walls of the barrel 104.

FIG. 2c illustrates an arrangement of the auto-injector 100 at the end of the injection. The connector 116 and the cartridge 110 have reached the limit of their range of motion. The administration device 117 guided by the connector 116 through the opening 115 has pierced the septum 109 of the cartridge 110, establishing fluid communication with the containment volume 122 comprising the beneficial agent 107. Because of the pressure differential between the pressurized pressure chamber 103 and containment volume 122 and the tissue of the subject, the stopper 106 is forced to move distally with respect to the barrel 104 of the cartridge 110, ejecting the beneficial agent 107 through the administration device 117 which injects the beneficial agent into the subject.

FIG. 3 illustrates an exploded view of the interface between the connector 116 and the cartridge 110. As noted above, FIG. 3 shows the connector body 114, the set of latch arms 112, the sealing surface 113, the flange 108, the cartridge barrel 104, and the locking ring 111. The piston 105 within the barrel 104 and the administration device 117 are partially occluded in the depiction provided in FIG. 3.

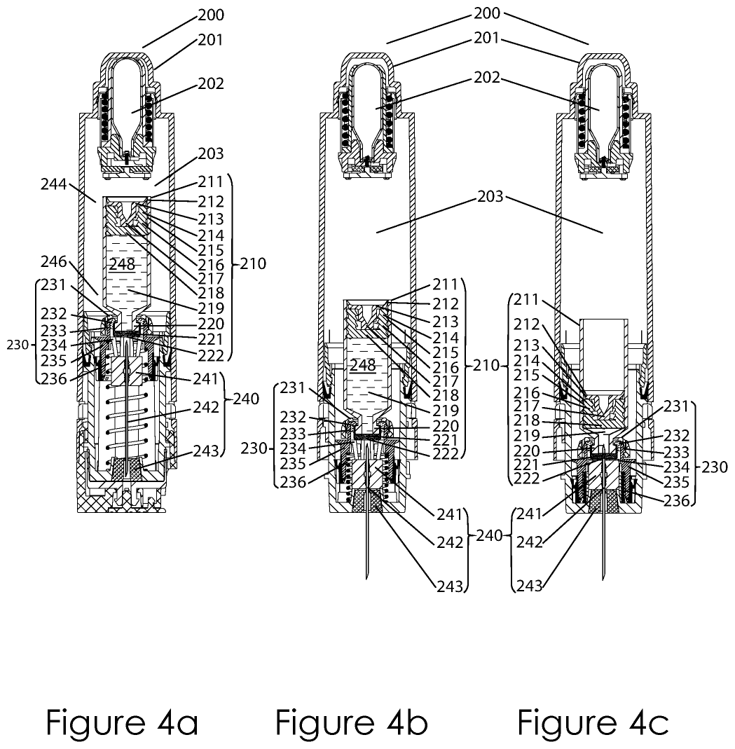

FIGS. 4a-4c illustrate another arrangement of an auto-injector 200 for administering a beneficial agent 219 to a subject. The auto-injector 200 comprises a housing 201 which may define a pressure chamber 203. An energy source 202 may be provided according to the discussion noted above in relation to FIGS. 2a-2c. FIGS. 4a-4c illustrate another example of a cartridge 210 that interfaces a connector 230 to dispose the cartridge 210 relative to an administration device 140.

The cartridge 210 disposed in a pressure chamber 203 and comprises a sidewall, which in the illustrated example may comprise a rigid cylindrical barrel 211 with a distal end 244 and a proximal end 246. The proximal end 246 is configured to expel the beneficial agent 219. The cartridge 210 further comprises a first stopper 216 moveably disposed in the barrel 211. The first stopper 216 includes a first seal surface 214 creating an interference region 215 against an inner surface of the barrel 211. The first seal surface 214 may create a fluid tight seal in the interference region 215. In addition, a containment volume 248 may be defined by the first stopper 216 and the inner surface of the barrel 211. The beneficial agent 219 may be disposed in the containment volume 248. The first stopper 216 additionally comprises an inner surface facing the beneficial agent 219 in the containment volume 248.

In some embodiments, the cartridge 210 comprises a second stopper 213 that is disposed in the barrel 211. The second stopper 213 may be disposed distal to the first stopper 216, but still within the barrel 211. The second stopper 213 may define a second seal surface 212. In addition, the second stopper 213 may comprise a post 217. The post 217 may be configured to displace a portion of the first stopper 216. The second seal surface 212 of the second stopper 213 interferes against the inner surface of the barrel 211 to create a fluid tight seal.

The proximal end of the cartridge 210 comprises a flange 220 having an opening sealed by a septum 221. The septum 221 may be disposed against said flange 220 and configured to seal the beneficial agent 219 inside the containment volume at the proximal end of the cartridge 210. The addition of the second stopper 213 may help maintain the orientation of the first stopper 216 within the barrel 211 as the first stopper 216 moves relative to the barrel 211 (e.g., when the beneficial agent 219 is expelled therefrom). Because the first stopper 216 and/or the second stopper 213 may be made of an elastomeric material that is ductile and flexible, the first stopper 216 and/or the second stopper 213 may experience varying levels of friction relative to the barrel 211 during movement along the inner surface of the barrel 211. As such, the first stopper 216 and/or the second stopper 213 may be prone to shifting out of an orientation perpendicular to the barrel 211. In turn, a loss of seal can occur. As such, a piston (e.g., as shown and described above in relation to FIGS. 2a-2c) may be provided that is more rigid than the first stopper 216 and/or the second stopper 213. In turn, the piston may stabilize the first stopper 216 and/or second stopper 218 as the first stopper 216 and/or second stopper 218 moves relative to the barrel 211 in a manner like that described above. In some embodiments, the interior surface of the barrel 211, the first stopper 216, the second stopper 218, and/or a piston may undergo siliconization, in order to lubricate surfaces which slide against one another, thus promoting their smooth relative movement.

In some embodiments, the cartridge 210 additionally comprises a crimp seal 222 surrounding the septum 221 and the flange 220 to secure the septum 221 against the opening of the cartridge 210.

As described above, the administration device 240 may include any one or more appropriate administration device to deliver the beneficial agent 219. In the depicted example, the administration device 240 may comprise a hub 241, a needle cannula 242, and a guide 243. The hub 241 comprises a hollow channel that may receive a portion of the needle cannula 242, which may be affixed to the hub 241. In turn, a first end and a second end of the needle cannula 242 may extend in opposite direction from the hub 241. The guide 243 may be attached to the auto-injector housing 101. The guide 243 may comprise a hollow channel that the needle cannula 242 may move freely within. The guide 243 serves as support for the needle cannula 242 limiting lateral motion of the needle cannula 242 to assist in aligning the needle cannula 242 with a longitudinal axis of the auto-injector 200 during activation.

In one embodiment, the first end of the needle cannula 242 may be configured to establish fluid communication with the beneficial agent 219 inside the cartridge 210. The second end of the needle cannula 242 may be configured to penetrate the subject's tissue. At the time of injection the beneficial agent 219 may, in response to an increase in pressure in the pressure chamber 203 relative to an ambient pressure, travel through the needle cannula 242 from the containment volume 248 of the cartridge 210 into the subject's tissue.

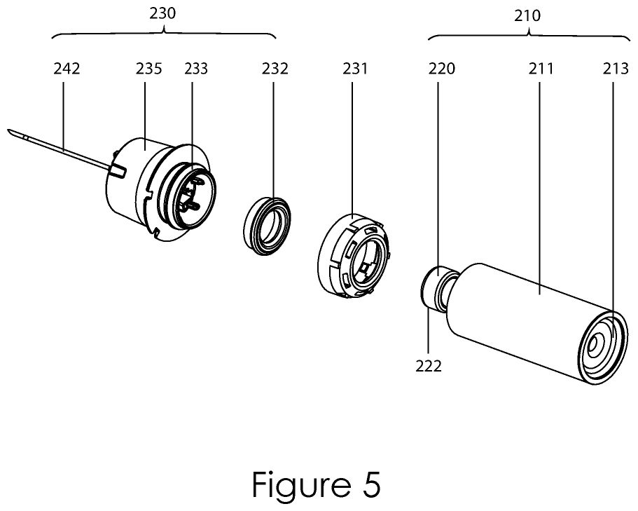

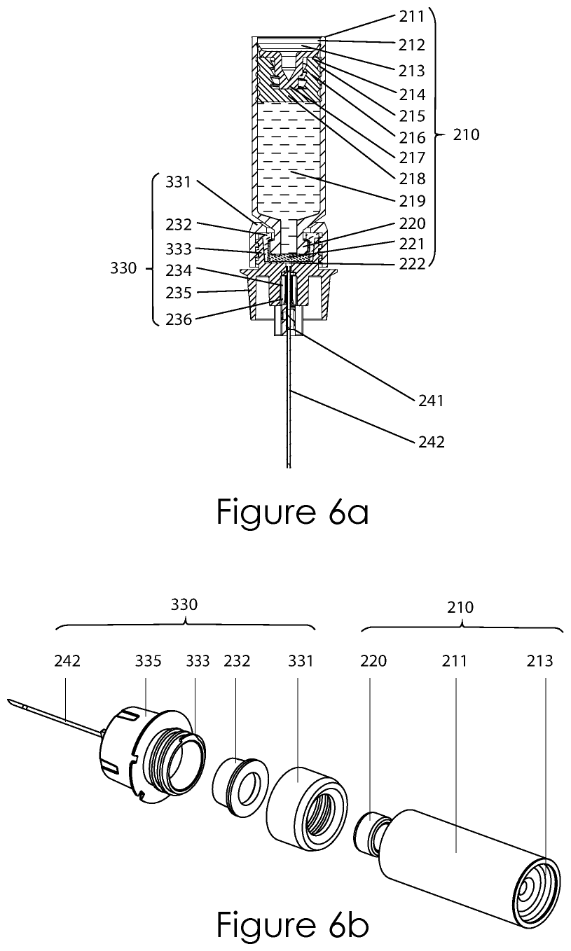

As shown in FIGS. 4a-4c and FIG. 5, the connector 230 may engage the cartridge 210 to maintain the cartridge relative to the administration device 240. The connector 230 may comprise a body 235 having a distal end and a proximal end. The connector 230 may include an opening 236 in the body 235 at the proximal end. The connector 230 may engage the administration device 240. A guide rib 234 may be provided that facilitates centering of the administration device 240 in the opening 236 of the connector 230. The guide rib 234 may also help to maintain the orientation of the administration device 240 as it travels towards the cartridge 210 during activation of the auto-injector 200. A gasket 232 may be provided that surrounds a portion of the flange 220 of the cartridge 210. The gasket 232 may abut the guide rib 234. In turn, the guide rib 234 and gasket 232 may bear against the flange 220 of the cartridge 210 to fix the location of the gasket 232 and cartridge 210 relative to the connector 230. A latch ring 233 may further support the gasket 232 by holding the gasket 232 against the cartridge 210. The latch ring 233 may extend in parallel relative to the barrel 211 of the cartridge 210. An outside surface of the latch ring 233 may be sloped such that the latch ring 233 gradually increases in diameter in a direction from the proximal end of the connector 230 towards a distal end of the connector 230. The latch ring 233 may include a step at a distal portion thereof. The step may comprise a smaller diameter than that of the latch ring 233 at its largest diameter to create a lip. The lip created in the latch ring 233 may be engaged by a complimentary feature provided on the locking ring 231 to latch the locking ring 231 to the latch ring 233. In an alternative example of a connector 330 shown in FIGS. 6a and 6b, rather than providing a lip on a latch ring 233, a latch ring 333 may be provided that includes a threaded portion with complimentary threads provided on a locking ring 331 to engage the locking ring 331 relative to the latch ring 333. Other components labeled with consistent reference numerals as those used in FIGS. 4a-5 may perform in a manner as described above.

In any regard, the locking ring 231 may be moved concentrically relative to the cartridge 210 to engage the latch ring 233. The locking ring 231 may interlock with the latch ring 233. In turn, the locking ring 231 may abut the gasket 232 to capture the gasket 232 between the locking ring 231 and the guide rib 234 to help maintain the gasket 232 in position relative to the cartridge 210. That is, the gasket 232 may be captured by the guide rib 234, latch ring 233, and locking ring 231 to maintain the gasket 232 in position relative to the cartridge 210.

The gasket 232 may be stretched to slip over the flange 220 of the cartridge 210. A shape of the gasket 232 may be complimentary to a shape of the flange 220 so as to include complimentary contoured surfaces that may be in contacting engagement when the gasket 232 is disposed on the flange 220.

The connector 230 may maintain engagement of the cartridge 210 with the auto-injector housing 201 to align and/or otherwise dispose the cartridge 210 in position relative to the administration device 240. The connector 230 may facilitate a secure coupling of the cartridge 210 relative to the auto-injector housing 201 and/or administration device 240 during an automated manufacturing process in which the auto-injector 200 is rendered into the pre-use configuration shown in FIGS. 2a. The connector 230 may also maintain the position of the cartridge 210 relative to the administration device 240 to facilitate fluid communication between the beneficial agent 219 in the containment volume 248 and the administration device 240 during activation of the auto-injector 200 (e.g., during injection of the beneficial agent 219).

At a proximal end, the connector 230 may include the opening 236 to accept the administration device 240 such the first end of the needle cannula 242 is disposed relative to the septum 221. As such, the first end of the needle cannula 242 may penetrate the septum 221 of the cartridge 210 to establish fluid communication between the needle cannula 242 and the containment volume 248. The guide rib 234 of the connector 230 may help provide orthogonal penetration of the first end of the needle cannula 242 through the septum 221.

The energy source 202 is configured to be activated when the auto-injector 200 is used to pressurize the pressure chamber 203. The needle cannula 242 is configured to transfer the beneficial agent 219 from the cartridge 210 to the subject in response to the pressurization of the pressure chamber 203. In some embodiments, the cartridge 210 may include one or more additional stoppers (e.g., a third stopper, which is not shown in the figures), thus creating a plurality of containment volumes for storing multiple beneficial agents. Alliteratively, a plurality of containment volumes may be provided for separately storing a dry (e.g., lyophilized or freeze dried) beneficial agent and a diluent for reconstitution of the dry beneficial agent at or near the time of activation of the auto-injector 200. As noted above, in such embodiments the barrel 211 may comprise at least one bypass (e.g., a wider section that permits fluid communication between adjacent compartments) when occupied by an intermediate stopper as described above.

The cartridge 210 disposed in the pressure chamber 203 is configured such that the pressure within the pressure chamber 203 is applied to the first stopper 216 and/or the second stopper 213 to pressurize the beneficial agent 219 in the containment volume 248. The pressure within the pressure chamber is also applied to an external surface of the barrel 211. In turn, because the pressure of the pressure chamber 203 is applied to the containment volume as a whole, very little or no pressure differential may occur between the containment volume 248 and the pressure chamber 103 such that there is no force applied across the barrel 211. That is, the pressure exerted by the beneficial agent 219 to an internal surface of the barrel 211 may be counteracted by a similar if not identical pressure acting on the external surface of the barrel 211. As such, very little force is applied to the barrel 211 while the beneficial agent 219 is expelled from the cartridge 210 through the administration device 240 upon activation of the auto-injector 200.

FIG. 4b illustrates an arrangement of the auto-injector 200 after it has been activated and before the injection of the beneficial agent 219 into a subject. The energy source 202 has pressurized the pressure chamber 203, driving the second end of the needle cannula 242 to a position in which the second end extends from the auto-injector housing 201 (e.g., and into the subject's tissue). The pressurization of the pressure chamber 203 also results in the cartridge 210 that is coupled to the administration device 240 by the connector 230 in a distal direction within the auto-injector housing 201. As the administration device 240 reaches the limit of an available range of motion within the auto-injector housing 201, the guide rib 243 may abut the needle hub 241 preventing further distal movement. However, the connector 230 and the cartridge 210 may continuing to travel towards the administration device 240 such that the first end of the needle cannula 242 pierces the septum 221 to establish fluid communication between the needle cannula 242 and the beneficial agent 219 in the containment volume 248.

A pressure differential between the pressure chamber 203 and an ambient pressure external to the auto-injector 200 may result in a force being applied to the second stopper 213. The second stopper 213 may transferring the force to the first stopper 216, which in turn may move relative to the barrel 211 to expel the beneficial agent 219 as the containment volume 248 is decreased. In some examples, the first stopper 216 may be provided without the second stopper 213 such that the force resulting from the pressure differential is applied directly to the first stopper 216. As noted above, pressure is also being applied at the exterior surface of the barrel 211, countering the pressure of the beneficial agent 219 on the interior surface of the barrel 211.

FIG. 4c illustrates an arrangement of the auto-injector 200 at the end of the injection. The connector 230 and the cartridge 210 have reached the limit of their range of motion. The administration device 240 guided by the connector 230 through the opening 236 has pierced the septum 221 (and in some embodiments through a crimp seal 222 of the cartridge 210) to establish fluid communication between the containment volume 248 and the administration device 240. Because of the pressure differential between the pressurized pressure chamber 203 and ambient pressure outside the pressure chamber 203 (e.g., in the tissue of the subject), the first stopper 216 and/or the second stopper 213 undergoes movement relative to the barrel 211 to decrease the containment volume 248 of the cartridge 210, thereby expelling the beneficial agent 219 through the administration device 240 (e.g., into the tissue of the subject).

FIG. 5 illustrates an exploded view of the connector 230 and the cartridge 210. The components of the connector 230 visible in FIG. 5 include the connector body 235, the latch ring 233, the gasket 232, and the locking ring 231. The components of the cartridge 210 visible in the figure are the barrel 211, the second stopper 213, the flange 220, and the crimp seal 222.

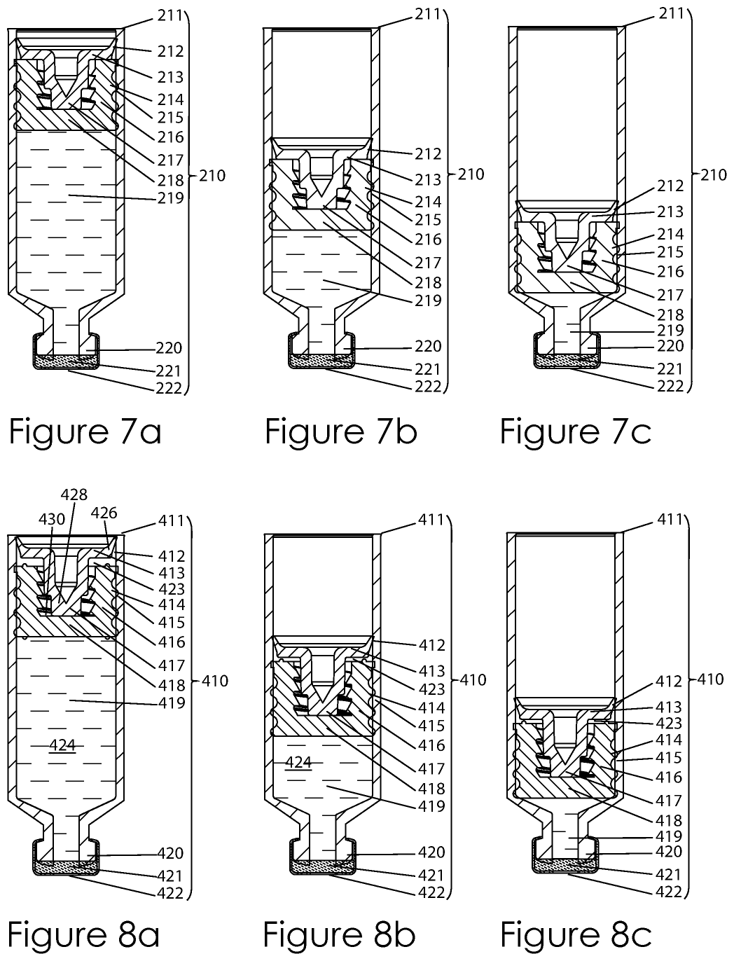

FIGS. 7a-7c illustrate a detailed view of the cartridge 210 expelling a beneficial agent 219 as described above in FIGS. 4a-4c.

FIGS. 8a-8c illustrate an alternative example of a cartridge 410 where friction between the interior walls of the reservoir and the first stopper disposed within the barrel is decreased to improve glide of the first stopper as it travels down the barrel. Specifically, a cartridge 410 may be provided generally according to the foregoing description of the cartridges 110 and 210. That, the cartridge 410 includes a sidewall 415 that may comprise a rigid cylindrical barrel. A flange 420 is provided with a septum 421 to close an opening in the flange 420 to seal an outlet of the cartridge 410. A crimp seal 422 may also be provided to assist in securing the septum 421 relative to the flange 420.

The cartridge 410 includes a containment volume 424 that may contain a beneficial agent 419. A first stopper 418 is provided with a first interference 414 relative to the sidewall 415 to provide sealing engagement between the first stopper 418 and the sidewall 415. A second stopper 413 may also be provided that may be arranged distally relative to the first stopper 418 The second stopper 413 may also define a second interface 412 between the second stopper 413 and the sidewall 415. The second interface 412 may provide sealing engagement between the second stopper 413 and the sidewall 415.

The second stopper 413 may include a post 417 that extends relative to a well 430 defined in the first stopper 418. The second stopper 413 also includes a skirt 426 that extends radially from the post 417 at a proximal portion of the second stopper 413. The distal portion of the post 417 may contacting engage the first stopper 418 such that the skirt 426 is offset from a proximal end portion of the first stopper 418. The offset between the skirt 426 and the first stopper 418 may create a gap 423 between the skirt 426 and the first stopper 418.

In turn, as a force acts on the second stopper 413 and the first stopper 418 to move the first stopper 418 and the second stopper 413 distally relative to the sidewall 415, the second stopper 413 may bear upon the first stopper 418 to cause deformation of the first stopper 418, thus reducing the gap 423 (e.g., the amount of offset) between the skirt 426 and the first stopper 418. The post 417 may also be configured in relation to the well 430 such that the relative distal movement of the second stopper 413 relative to the first stopper 418 may cause radial contraction of the first stopper 418. The first stopper 418 may be axially stretched as well. In any regard, the radial contraction of the first stopper 418 results in a reduction of a normal force acting between the first stopper 418 and the sidewall 415 at the first interference 414. In turn, a friction force acting to withstand distal movement of the first stopper 418 may be reduced and the first stopper 418 may be advanced distally with lower friction, thus promoting efficient and smooth travel of the first stopper 418 relative to the sidewall 415 to dispense the beneficial agent 419. As such, when the first stopper 418 and the second stopper 413 are stationary (e.g., in the absence of a force advancing the first stopper 418 and the second stopper 413), the first interference 414 provides an enhanced seal that may be effective to provide an aseptic barrier seal for storage of the cartridge 410. However, upon influence of a force advancing the first stopper 418 and the second stopper 413 (e.g., during activation of an auto-injector), the force acting normally between the first stopper 418 and the sidewall 415 may be reduced, thus allowing the first stopper 418 to be more easily advanced along the sidewall 415. As the cartridge 410 may be arranged as described above within a pressure chamber, equalized pressure on either side of the sidewall 415 may help ensure a seal is maintained even as the first stopper 418 is advanced with lower normal forces acting to create a seal.

The first stopper 418 and the second stopper 413 may be biased apart by a biasing member. A biasing member such as a spring (not shown) may be provided between the first stopper 418 and the second stopper 413 to maintain the first stopper 418 and the second stopper 413 is spaced or disengaged relation. Alternatively, the biasing member may be facilitated by an elasticity of either or both of the first stopper 418 and the second stopper 413

While the cartridge 410 illustrated in FIGS. 8a-8c may be used in an auto-injector activated through increased pressure in a pressure chamber as described above, the arrangement of the first stopper 418 and second stopper 413 may also be used in an arrangement in which the stoppers 413/418 are advanced under the influence of other forces such as a mechanical force acting on the stoppers 413/418. For instance, the second stopper 413 may be engaged by a rod or plunger to create the distal movement relative to the first stopper 418 to achieve operation as described above. As such, the cartridge 410 arrangement shown in FIGS. 8a-8c may be used with an auto-injector as described above. In other examples, the configuration of the first stopper 418 and second stopper 413 may also be provided for other containers, syringes, cartridges, barrels, or other sidewall such that the arrangement may be used in conjunction with other apparatuses such as a pump, a syringe, or other dispensing systems.

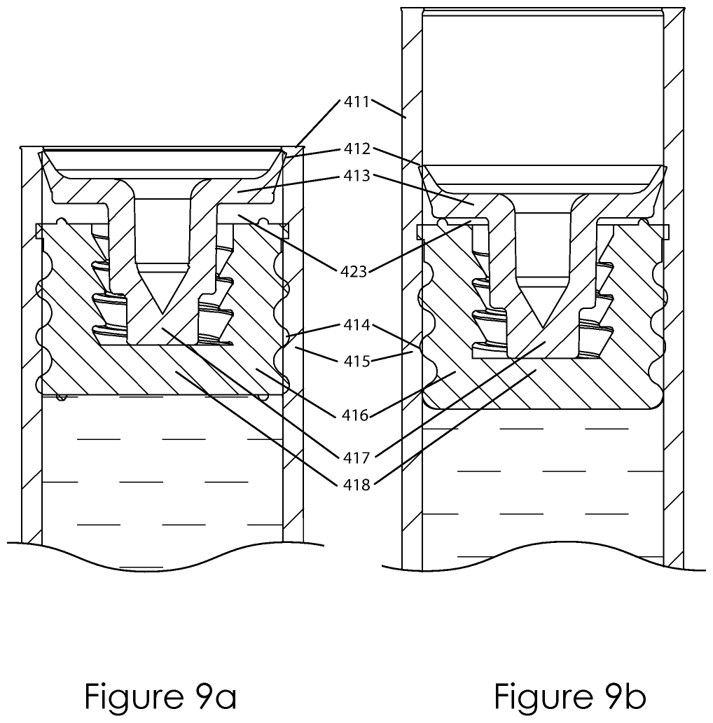

FIGS. 9a-9b provide magnified views of the portions of FIGS. 8a-8b showing the interaction of the stoppers 413/418 before and during auto-injector activation. Upon activation of the auto-injector, the post 417 of the second stopper 413 deforms the first stopper 418, stretching said stopper axially and contracting it radially, to reduce the first interference 414, reducing friction between the first stopper 148 and the sidewall 415.

While the invention has been illustrated and described in detail in the drawings and foregoing description, such illustration and description is to be considered as exemplary and not restrictive in character. For example, certain embodiments described hereinabove may be combinable with other described embodiments and/or arranged in other ways (e.g., process elements may be performed in other sequences). Accordingly, it should be understood that only the preferred embodiment and variants thereof have been shown and described and that all changes and modifications that come within the spirit of the invention are desired to be protected.

What is claimed is:

1. An auto-injector for administering a beneficial agent, the auto-injector comprising:
a pressure chamber;
a primary container comprising a barrel defining a rigid sidewall with an inner surface and an exterior surface, wherein the barrel is disposed within the pressure chamber;
a stopper disposed in the barrel and sealingly engaged with the inner surface to define a containment volume within the primary container, the containment volume comprising a beneficial agent; and
wherein an increase in pressure within the pressure chamber results in an increase in a first pressure in the pressure chamber acting uniformly on the stopper and the exterior surface to increase a second pressure in the containment volume creating a pressure differential relative to ambient pressure of an exterior environment of the auto-injector to cause the stopper to move relative to the inner surface to reduce the containment volume and expel the beneficial agent from the primary container.

2. The auto-injector of claim 1, further comprising:
an administration device disposed relative to the primary container to establish fluid communication with the containment volume, wherein the beneficial agent is expelled through the administration device.

3. The auto-injector of claim 2, wherein the primary container comprises a cartridge, and the auto-injector further comprises:
a connector engaged with the cartridge to dispose the containment volume relative to the administration device.

4. The auto-injector of claim 3, wherein the connector further comprises:
a connector body having a distal end and proximal end;
an opening in the connector body at the proximal end that is configured for receiving the administration device; and
a sealing surface surrounding the opening and disposed between the connector body and the cartridge to provide a seal between the cartridge and the connector.

5. The auto-injector of claim 4, wherein the connector further comprises:
a plurality of latch arms at the distal end configured to engage a flange of the cartridge, wherein the latch arms are displaceable radially to receive the flange; and
a locking ring positionable relative to the plurality of latch arms to prevent radial displacement of the plurality of latch arms.

6. The auto-injector of claim 1, further comprising:
an energy source that is selectively activatable to cause the increase in pressure within the pressure chamber.

7. The auto-injector of claim 1, wherein the first pressure in the pressure chamber equals the second pressure in the containment volume.

8. The auto-injector of claim 1, further comprising:
a piston disposed in the barrel between the stopper and the pressure chamber, the piston defining a supplementary sealing interface with the inner surface of the barrel, wherein the supplementary sealing interface is provided between the stopper and the pressure chamber.

9. An auto-injector for administering a beneficial agent to a subject, the auto-injector comprising:
a housing comprising a pressure chamber;
a cartridge disposed in the pressure chamber comprising:
a barrel comprising a rigid cylindrical sidewall, a distal end and a proximal end, the proximal end being configured to expel the beneficial agent,
a first stopper moveably disposed in the barrel and sealing against the sidewall and defining a containment volume between the first stopper and the proximal end of the barrel, and
a beneficial agent disposed in the containment volume;
a pressure source configured to be activated when the auto-injector is used and to pressurize the pressure chamber;
an administration device configured to transfer the beneficial agent from the cartridge and to administer it to the subject; and
the cartridge being disposed in the pressure chamber such that pressure is directed to the stopper to pressurize the beneficial agent in the containment volume, and to an external side of the sidewall to counter pressure exerted by the beneficial agent to an internal side of the sidewall when the beneficial agent is expelled from the cartridge through the administration device.

10. The auto-injector of claim 9, wherein the cartridge further comprises a second stopper moveably disposed in the barrel between the first stopper and the distal end of the cartridge and sealing against the sidewall, preventing fluid in the pressure chamber from reaching the first stopper.

11. The auto-injector of claim 9, wherein the pressure source further comprises one of compressed gas or liquefied gas.

12. The auto-injector of claim 9, wherein the administration device is one of a needle, a micro-needle, an array of needles, a jet injector nozzle, a nasal nozzle, a dispenser, or a connector.

13. A connector for a cartridge comprising a beneficial agent, the connector comprising:
a body having a distal end and a proximal end;
an opening in the body at the proximal end configured for engaging an administration device;
a sealing surface surrounding said opening;
an engagement feature at the distal end configured to couple to a flange of the cartridge;
a locking ring;
the cartridge comprising:
a rigid barrel comprising a cylindrical sidewall, a distal end portion and a proximal end portion, the proximal end portion comprising an opening and the flange;
a first stopper moveably disposed in the barrel and sealing against the sidewall to define a containment volume between the stopper and the proximal end portion for storing the beneficial agent;
a septum disposed against said flange and configured to seal the beneficial agent inside the containment volume at the proximal end portion;
wherein the engagement feature of the connector is configured to engage the flange of the cartridge to couple the connector to the cartridge; and
wherein the locking ring surrounding the engagement feature when coupled to the cartridge and preventing the engagement feature from disengaging from the flange.

14. A cartridge for storing in, and delivering from a beneficial agent, the cartridge comprising
a cylindrical sidewall;
a dispensing port at a first end;
a first stopper moveably disposed relative to the sidewall and offset from the dispensing port, the stopper engaging with the sidewall wall to establish a fluid tight seal therebetween;
a beneficial agent accommodated between the dispensing port and the stopper in a containment volume;
a second stopper moveably disposed relative to the sidewall and disposed on a side of the first stopper opposite the dispensing port, the second stopper engaged with the sidewall to establish a fluid tight seal therebetween; and
wherein upon application of a force to the second stopper to cause advancement of the second stopper relative to the sidewall, the second stopper advances relative the first stopper to radially contract the first stopper to reduce a normal force acting between the first stopper and the sidewall such that the first stopper and the second stopper move together relative to the dispensing port to expel the beneficial agent through the dispensing port.

15. The cartridge of claim 14 wherein the first stopper comprises
a seal region extending relative to the sidewall; and
a well configured such than when the second stopper moves relative the first stopper it displaces the well causing the seal region to stretch.

16. The cartridge of claim 14 wherein the second stopper comprises a seal region and a post, the post is configured to displace a portion of the first stopper.

17. The cartridge of claim 14, wherein in a biasing member is disposed between the first stopper and the second stopper to bias apart the first stopper and the second stopper.

18. The cartridge of claim 17 wherein the biasing member is provided by an elasticity of at least one of the first stopper and the second stopper.
